(19) United States
(12) Patent Application Publication
(10) Pub. No.: US 2019/0209961 A1
(43) Pub. Date: Jul. 11, 2019

(54) Carbon Dioxide Capture Systems

(71) Applicant: Mote, Inc., Los Angeles, CA (US)

(72) Inventors: Patrick Kennedy, Los Angeles, CA (US); Harshul Thakkar, Douglasville, GA (US)

(21) Appl. No.: 16/243,022

(22) Filed: Jan. 8, 2019

Related U.S. Application Data
(60) Provisional application No. 62/615,296, filed on Jan. 9, 2018.

(51) Int. Cl. B01D 53/04; C02F 3/32; C12P 1/00; C10L 1/02; B01J 20/34; C05F 11/00

(52) U.S. Cl. CPC B01D 53/0415; B01D 53/0446; B01D 53/0438; C02F 3/322; C12P 1/00; C10L 2200/0484; B01J 20/3483; C05F 11/00; B01D 2257/504; B01D 2259/4566; B01D 2259/40088; C10L 1/02

(57) ABSTRACT

Carbon dioxide capture systems in accordance with various embodiments of the present disclosure are provided. In one embodiment, an apparatus for capturing carbon emissions from a vehicle is provided, comprising: an exhaust feeder comprising a first end and a second end, wherein the first end connects to the vehicle and receives exhaust gases emitted from the vehicle; a housing connected to the second end and secured to an exterior surface of the vehicle, wherein the housing receives the exhaust gases emitted from the vehicle via the exhaust feeder; wherein the housing comprises: at least one gas deflector surface that directs the exhaust gases to a sorbent material configured to capture carbon dioxide from the exhaust gases; a detachable section that allows for removal and installation of the sorbent material; and an exit port that allows for release of residual gases after the sorbent material captures carbon dioxide.

### CROSS-REFERENCE TO RELATED APPLICATION

This application claims priority to provisional application Ser. No. 62/615,296, filed on Jan. 8, 2018, the entire contents of which are hereby incorporated by reference.

### FIELD OF THE INVENTION

The present disclosure generally relates to capturing carbon dioxide and more specifically to carbon dioxide capture systems for vehicles.

### BACKGROUND

A major driver of human-caused climate change is the release of carbon dioxide (may also be referred to as “CO₂”) and other pollutants by vehicles (may also be referred to as “motor vehicles”). For example, with each gallon of gasoline and diesel generating 9 to 10 kilograms of CO₂ and 99.9% of U.S. school buses still using fossil fuels, school buses have generated more than 9 million tons of CO₂ into the atmosphere. Overall, U.S. vehicles burned over 140 billion gallons of gasoline and about 40 billion gallons of diesel in 2016, resulting in massive releases of CO₂. Considering emissions by motor vehicles contribute to many of the problems of climate change, it is likely that laws in the future will require progressively lower greenhouse gas emissions. Transit authorities, in anticipation of new regulation, have already introduced electric or plug-in hybrid electric buses. Yet many types of vehicles, such as electric or hybrid electric school buses, tend to be extremely expensive, of limited battery life, and are thus not economically feasible for school districts in need of resources.

### SUMMARY OF THE INVENTION

The various embodiments of the carbon dioxide capture system contain several features, no single one of which is solely responsible for their desirable attributes. Without limiting the scope of the present embodiments, their more prominent features will now be discussed below. In particular, the present carbon dioxide capture systems will be discussed in the context of CO₂ and particular sorbents for capturing CO₂ from exhaust gases of vehicles. However, the particular configurations discussed are merely exemplary and various other configurations may be utilized as appropriate to the requirements of a specific application in accordance with various embodiments of the invention. For example, carbon dioxide capture systems may be configured to capture various other pollutants such as (but not limited to) methane (CH₄), nitrous oxide (N₂O), fluorinated gases (e.g., hydrofluorocarbons, perfluorocarbons, sulfur hexafluoride, nitrogen trifluoride, etc.) and/or ozone-depleting substances (e.g., chlorofluorocarbons, hydrochlorofluorocarbons, halons, etc.). In some embodiments, the sorbent material may be specific to the type of pollutant for capture. Further, although carbon dioxide capture systems will be discussed in the context of capturing gases from vehicles, the present carbon dioxide capture systems may be utilized to capture pollutants from any source. After considering this discussion, and particularly after reading the section entitled “Detailed Description,” one will understand how the features of the present embodiments provide the advantages described here.

One aspect of the present embodiments includes the realization that carbon dioxide capture system needs to be easily mounted, serviced, and replaced. The present embodiments solve this problem by introducing a housing that is readily securable with a vehicle, as further described below. Further, the housing may include a sorbent material that is readily removable. The present embodiments thus advantageously enable ease in serviceability. The present embodiments provide these advantages and enhancements, as described below.

In a first aspect, an apparatus for capturing carbon emissions from a vehicle, comprising: an exhaust feeder having a first end and a second end, wherein the first end of the exhaust feeder connects to the vehicle and receives exhaust gases from the vehicle; a housing connected to the second end of the exhaust feeder and secured to an exterior surface of the vehicle, wherein the housing receives exhaust gases from the vehicle via the exhaust feeder and comprises at least one gas deflector surface that directs exhaust gases to a sorbent material to capture carbon dioxide from exhaust gases, a detachable section that allows for removal and installation of the sorbent material, and an exit port that allows for release of residual gases.

In an embodiment of the first aspect, the first end of the exhaust feeder may connect to the vehicle via a tailpipe of the vehicle.

In another embodiment of the first aspect, the exhaust feeder may be constructed using heat-resistant and corrosion-resistant materials.

In another embodiment of the first aspect, the housing may be constructed using heat-resistant and corrosion-resistant materials.

In another embodiment of the first aspect, the housing may be secured to a top, a bottom, or a rear of the vehicle.

In another embodiment of the first aspect, the housing may further comprise a plurality of mesh bags configured to hold the sorbent material while still allowing the exhaust gases directed from the gas deflector surfaces to pass through the sorbent material.

In another embodiment of the first aspect, the apparatus may further comprise a flange that secures the exhaust feeder to the housing using a locking mechanism.

In another embodiment of the first aspect, the apparatus may further comprise a flange that secures the exhaust feeder to the housing using a slotting mechanism.

In another embodiment of the first aspect, the exhaust feeder may be constructed using a flexible material.

In another embodiment of the first aspect, the gas deflector surfaces may be located in the housing and centered around the exhaust feeder.

In a second aspect, a method for reducing carbon emissions from a vehicle, comprising: receiving exhaust gases emitted from a vehicle at a first end of an exhaust feeder; delivering exhaust gases from the first end of the exhaust feeder to a second end of the exhaust feeder; transporting the exhaust gases from the second end of the exhaust feeder into a housing; directing the exhaust gases in the housing to a sorbent material using gas deflector surfaces; adsorbing carbon dioxide from the exhaust gases using the sorbent material; expelling residual gases using an exit port in the housing after the sorbent material captures carbon dioxide from the exhaust gases; removing a section of the housing to allow for extraction of the sorbent material; heating the sorbent material after extraction to regenerate the sorbent material; storing carbon dioxide expelled from the sorbent material during heating of the sorbent material in a storage tank; utilizing the carbon dioxide for algae development including usage in biofuels, biomass generation, wastewater treatment, and fertilizers; and reinstalling the sorbent material in the housing after regeneration of the sorbent material from heating.

In an embodiment of the second aspect, the first end of the exhaust feeder may connect to the vehicle via a tailpipe of the vehicle.

In another embodiment of the second aspect, the exhaust feeder may be constructed using heat-resistant and corrosion-resistant materials.

In another embodiment of the second aspect, the housing may be constructed using heat-resistant and corrosion-resistant materials.

In another embodiment of the second aspect, the housing may be secured to a top, a bottom, or a rear of the vehicle.

In another embodiment of the second aspect, the housing may further comprise a plurality of mesh bags configured to hold the sorbent material while still allowing the exhaust gases directed from the gas deflector surfaces to pass through the sorbent material.

In another embodiment of the second aspect, a flange may secure the exhaust feeder to the housing using a locking mechanism.

In another embodiment of the second aspect, a flange may secure the exhaust feeder to the housing using a slotting mechanism.

In another embodiment of the second aspect, the exhaust feeder may be constructed using a flexible material.

In another embodiment of the second aspect, the gas deflector surfaces may be located in the housing and centered around the exhaust feeder.

### BRIEF DESCRIPTION OF THE DRAWINGS

The various embodiments of the present invention now will be discussed in detail with an emphasis on highlighting the advantageous features. These embodiments depict the novel and non-obvious embodiments shown in the accompanying drawings, which are for illustrative purposes only. These drawings include the following figures:

FIG. 1 illustrates a carbon dioxide capture system secured to a vehicle in accordance with an embodiment of the invention.

FIG. 2 illustrates a cargo box (may also be referred to as “housing”) of the carbon dioxide capture system in accordance with an embodiment of the invention.

FIG. 3 illustrates a carbon dioxide capture system secured to a vehicle in accordance with another embodiment of the invention.

FIG. 4A is a diagram illustrating a cartridge comprising meshes in accordance with an embodiment of the invention.

FIG. 4B is a diagram illustrating a cartridge without meshes in accordance with an embodiment of the invention.

FIG. 4C is a top-down perspective diagram illustrating a cartridge comprising meshes in accordance with an embodiment of the invention.

FIG. 4D is a top-down perspective diagram illustrating a cartridge comprising meshes and sorbent in accordance with an embodiment of the invention.

FIG. 5 is a flowchart illustrating a process for reducing CO₂ emissions from a vehicle using a carbon dioxide capture system in accordance with an embodiment of the invention.

FIG. 6 is a flowchart illustrating a process for capturing CO₂ in accordance with an embodiment of the invention.

FIG. 7 is a flowchart illustrating a process for regenerating a sorbent material in accordance with an embodiment of the invention.

FIG. 8 is a flowchart illustrating a process for reducing CO₂ emissions from a vehicle using a carbon dioxide capture system in accordance with another embodiment of the invention.

### DETAILED DESCRIPTION OF THE DRAWINGS

The following detailed description describes the present embodiments with reference to the drawings. In the drawings, reference numbers label elements of the present embodiments. These reference numbers are reproduced below in connection with the discussion of the corresponding drawing features. These drawings, and their written descriptions, indicate that certain components of the apparatus are formed integrally, and certain other components are formed as separate pieces. Those of ordinary skill in the art will appreciate that components shown and described herein as being formed integrally may in alternative embodiments be formed as separate pieces. Those of ordinary skill in the art will further appreciate that components shown and described herein as being formed as separate pieces may in alternative embodiments be formed integrally. Further, as used herein the term integral describes a single unitary piece.

In many embodiments, carbon dioxide capture system may be secured to a vehicle and configured to receive exhaust gases from the vehicle as further described below. In various embodiments, the carbon dioxide capture system may include an exhaust feeder configured to connect to the vehicle (e.g., via a vehicle's tailpipe) and receive exhaust gases emitted by the vehicle during operation. In several embodiments, the carbon dioxide capture system may include a housing containing sorbent material (e.g., mesh bags of sorbent) for capturing carbon dioxide and/or any other pollutant from the exhaust gases. In many embodiments, after the carbon dioxide has been adsorbed from the exhaust gases, the carbon dioxide capture system may release residual gases via an exit port. When the sorbent material is saturated following the adsorption of carbon dioxide, it may be extracted and then regenerated for continual use. For example, sorbent material may be regenerated using elevated temperatures (e.g., microwaving at 100-130 degrees Celsius) and/or applying reduced pressure. In many embodiments, the regeneration process may also expel the carbon dioxide adsorbed in the sorbent material for use in environmentally friendly consumption. For example, the captured carbon dioxide may be expelled from the sorbent material and stored for carbon fixation (e.g., photosynthesis) to promote algae development. Algae may be used to generate biofuels, biomass, wastewater treatment substances, and fertilizers. In many embodiments, after regeneration, the sorbent material may be reinstalled into the carbon dioxide capture system. As described further below, various embodiments and/or configurations of the carbon dioxide capture systems may be used in accordance with specific conditions or applications of a vehicle. For example, the carbon dioxide capture system may be configured for various sorbent capacity, structural configuration, material usage, etc. The embodiments discussed in accordance with the carbon dioxide capture systems help to solve current issues of carbon capture from vehicles, such as limitations of space, increased costs of equipment, and vehicle fuel economy. Carbon dioxide capture systems using cargo boxes in accordance with embodiments of the invention, are discussed below.

### Carbon Dioxide Capture Systems Using Cargo Boxes

Carbon dioxide capture systems may include so called cargo boxes (may also be referred to as “housing”). In some embodiments, a cargo box may be an off-the-shelf cargo box that may be customized for use with the carbon dioxide capture system. Although the term cargo box is used herein, the cargo box may not actually allow for storing cargo but instead may include various components for capturing gases (e.g., CO₂), as further described below. Further, the carbon dioxide capture system may also include various components connected to the cargo box, as further described below.

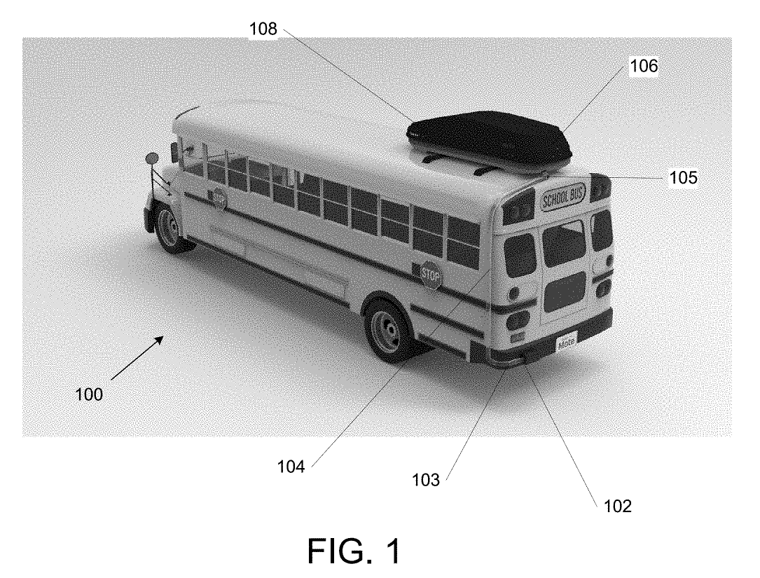

A carbon dioxide capture system secured to a vehicle in accordance with an embodiment of the invention is illustrated in FIG. 1. The carbon dioxide capture system may include an exhaust feeder 104 that receives exhaust gases from a tailpipe 102 of a vehicle 100 (e.g., bus). In many embodiments, the exhaust feeder 104 may include a first end 103 and a second end 105, where the first end 103 of the exhaust feeder 104 may connect to the tailpipe 102 to receive exhaust gases emitted from the vehicle 100. The exhaust feeder 104 may then deliver the exhaust gases to a cargo box 106 that captures carbon dioxide from the exhaust gases using sorbents (may also be referred to as “sorbent material(s)”), as further described below. In various embodiments, the cargo box 106 may be connected to the second end 105 of the exhaust feeder 104. In many embodiments, residual gases that remain after carbon dioxide has been captured from the exhaust gases may exit the cargo box 106 via an exit port 108 and into the environment. For example, the exit port 108 may be located on the cargo box 106 at an opposite end of the exhaust feeder 104 so that exhaust gases pass through materials in the cargo box 106 before exiting via the exit port 108.

In several embodiments, the cargo box 104 may be secured to the vehicle 100 in a manner known to one of skill in the art. For example, the cargo box 106 may be secured to a top, bottom, front, or rear of a vehicle, provided that placement of the cargo box 106 does not significantly impair control or driving of the vehicle. In some embodiments, the cargo box 104 may be secured within the vehicle 100. Considering the cargo box 106 will house exhaust gases that may be corrosive and/or of a high temperature, the cargo box 106 may also be constructed using heat-resistant and/or corrosion-resistant material, such as (but not limited to) metals, stainless steel, titanium, high-temperature plastics, and/or silicon carbide. Likewise, the exhaust feeder 104 may be constructed using heat-resistant and/or corrosion-resistant materials. Furthermore, the exhaust feeder 104 may be also constructed using a flexible material and may take the shape of a tube and/or pipe to adapt to the surfaces of different vehicles and placement of the cargo box 106 on the vehicle 100. For example, the exhaust feeder 104 may bend in accordance to the surface of a truck if the cargo box 106 is placed in a rear of the truck.

As further described below, the cargo box may include at least one gas deflector surface that directs the exhaust gases to a sorbent configured to capture carbon dioxide from the exhaust gases. In various embodiments, a sorbent may be porous materials capable of capturing carbon dioxide from gases or exhaust streams. The captured carbon dioxide is often removed afterwards by elevated temperatures and/or applying reduced pressure and then stored for use in biological carbon fixation, such as use of carbon dioxide to achieve algae growth, as further described below. Sorbents may include metal-organic frameworks (MOFs), zeolites, silica, calcium oxides, organic-inorganic hybrids, alkylamines/amines, hydrotalcites, and activated carbons. The many types of sorbents vary in performance. Examples of common MOFs include MOF-74, UTSA-16, MIL-101, and HKUST-1, while examples of zeolite structures include FAU, LTA, MFI, and CHA. Both MOFs and zeolites provide a high capture rate of carbon dioxide but drastically degrade in humid environments, a trait common to areas of school transportation. Hydrotalcites and metal-oxides are also known for a high capture rate but require high temperatures in excess of 300 degrees Celsius. Activated carbons provide a high capture rate in both dry and humid environments. Overall, effective sorbents may capture 20 percent of initial mass in carbon dioxide. A person of ordinary skill in the art will appreciate the differences in sorbent material and select suitable sorbent material in accordance with varying operating conditions of vehicles. For reference, Table 1 below is a list of some available sorbents.

TABLE 1

| Name | Type |
|---|---|
| PEI-MIL-101 | Amine-MOF |
| mmen-Mg₂(dobpdc) | Amine-MOF |
| dmen-Mg₂(dobpdc) | Amine-MOF |
| dmpn-Mg₂(dobpdc) | Amine-MOF |
| mmen-CuBTTri | Amine-MOF |
| NH₂-MIL-53(Al) | Amine-MOF |
| en-CuBTTri | Amine-MOF |
| Mg-MOF-74 | MOF |
| Ni-MOF-74 | MOF |
| Co-MOF-74 | MOF |
| HKUST-1 | MOF |
| SIFSIX-3(Zn) | MOF |
| Zn(ox)(atz)₂ | MOF |
| Zn-MOF-74 | MOF |
| CuTATB-60 | MOF |
| bio-MOF-11 | MOF |
| FeBTT | MOF |
| MOF-253-Cu(BF4) | MOF |
| ZIF-78 | MOF |
| CuBTTri | MOF |
| SNU-50 | MOF |
| USO-2-Ni-A | MOF |
| MIL-53(Al) | MOF |
| MIL-47 | MOF |
| UMCM-150 | MOF |
| MOF-253 | MOF |
| ZIF-100 | MOF |
| MTV-MOF-EHI | MOF |
| ZIF-8 | MOF |
| IRMOF-3 | MOF |
| MOF-177 | MOF |
| UMCM-1 | MOF |
| MOF-5 | MOF |
| 13X | Zeolite |
| Ca-A | Zeolite |

Although specific carbon dioxide capture systems are discussed with respect to FIG. 1, any of a variety of carbon dioxide capture systems as appropriate to the requirements of a specific application can be utilized in accordance with embodiments of the invention. Cargo boxes for use in carbon dioxide capture systems in accordance with the invention are further discussed below.

### Cargo Boxes

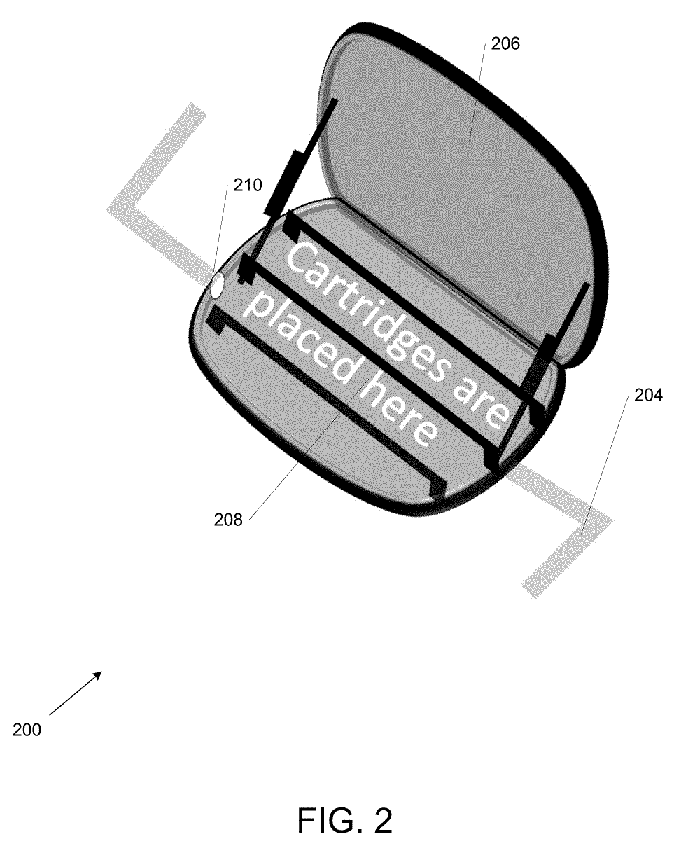

Carbon capture systems may include cargo boxes that include various components such as (but not limited to) an input port connected to an exhaust feeder, gas defector surface(s), a detachable section that allows for removal and installation of sorbent material(s), an exit port. A cargo box of a carbon dioxide capture system in accordance with an embodiment of the invention is illustrated in FIG. 2. As described above, a first end of an exhaust feeder 204 may connect to a tailpipe of a vehicle and allow for exhaust gases emitted from the vehicle to be received by a cargo box 200. In many embodiments, the cargo box 200 may include an inner section 208 (may also be referred to as “detachable section”) where one or more cartridge(s) containing sorbent material may be placed. In various embodiments, the detachable section allows for removal and installation of the sorbent material.

In many embodiments, the cartridge(s) may be a container for the sorbents thereby allowing the removal and installation of the sorbent by removing and installing the cartridges. In this manner, the sorbents may be contained and handled using the cartridges. Further, the cartridges may be constructed using various materials suitable for handling sorbents and/or exhaust gases. In some embodiments, the cartridges may be one or more mesh bag(s) configured to hold the sorbent. In such embodiments, the mesh bags may allow the exhaust gases directed from the gas deflector surface(s) to pass though the sorbent material. In some embodiments, the cartridges may be built into the detachable section 208 of the cargo box 200. In some embodiments, the cartridges may be placed in any configuration provided exhaust gases enter the cargo box 200 via the input port and residual gases exit via an exit port 210. In some embodiments, each of the cartridges 208 may have its own input port and exit port.

In further reference to FIG. 2, when the sorbents are partially or fully saturated following capture of carbon dioxide, the cargo box 200 may be opened by opening a lid 206 to allow for extraction of the cartridges from the detachable section 208. Mechanisms for opening and closing the cargo box 200 may include that of a cover-and lid mechanism or a locking and/or slotting mechanism or any other mechanism known by a person of ordinary skill in the art. The placement of the cargo box 200 and the materials used in construction of the cargo box 200 may be determined based on the specific needs of an application and known by a person of ordinary skill in the art. For example, the cargo box 200 may be constructed using metals, stainless steel, titanium, high-temperature plastics, silicon carbide, etc.

Although specific cargo boxes of carbon dioxide capture systems are discussed with respect to FIG. 2, any of a variety of cargo boxes as appropriate to the requirements of a specific application can be utilized in accordance with embodiments of the invention. Carbon dioxide capture systems using cartridges in parallel and/or in series in accordance with the invention are discussed below.

Carbon Dioxide Capture Systems Using Cartridges in Parallel and/or in Series

Carbon dioxide capture systems may be configured using cartridges in parallel and/or in series, as further described below. In many embodiments, the cartridges may or may not use a cargo box configuration but may instead be connected using a manifold and/or a coupler. For example, the cartridges may be connected to each other in parallel using a manifold. In a further example, cartridges may be connected to each other in series using a coupler, as further described below.

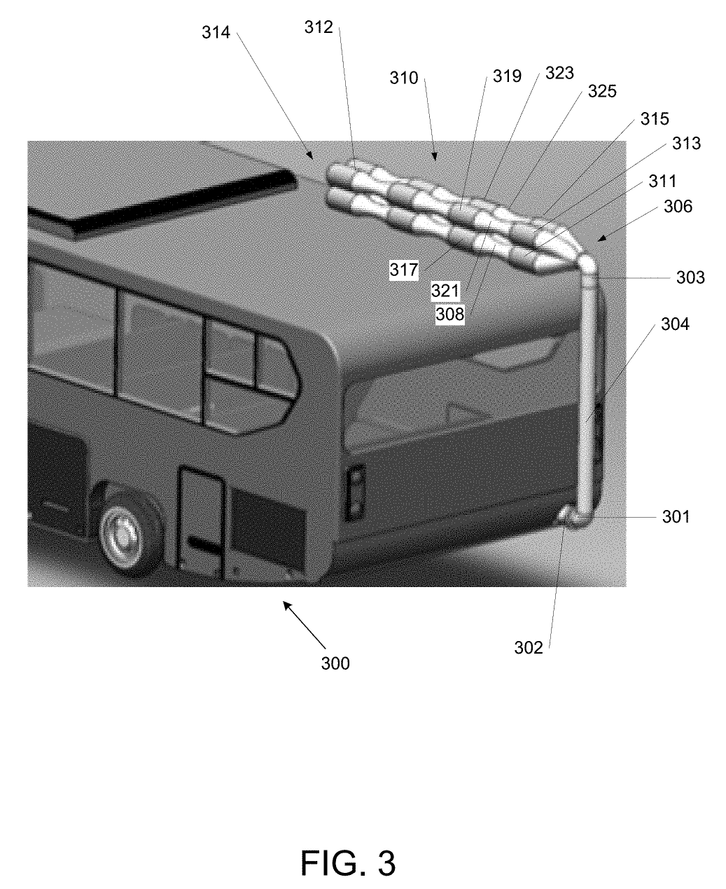

A carbon dioxide capture system secured to a vehicle in accordance with another embodiment of the invention is illustrated in FIG. 3. The carbon dioxide capture system may include an exhaust feeder 304 having a first end 301 and a second end 303. As described above, the first end 301 may connect to a tailpipe 302 of a vehicle 300 and receives exhaust gases emitted by the vehicle 300 (e.g., bus). The exhaust feeder 304 may then deliver the exhaust gases to a manifold 306. In many embodiments, the manifold 306 connects to a plurality of cartridges 310 that are in parallel with each other (e.g., cartridges 311, 313, 315). Connection in parallel of the cartridges 311, 313, 315 may allow for greater sorbent capacity such that exhaust gases are exposed to a larger volume of sorbent materials. For example, exhaust gases from the exhaust feeder 304 may be distributed to the cartridges 311, 313, 315 via the manifold 306. As described above, the cartridges 310 (e.g., cartridges 311, 313, 315) may contain sorbent material, such that when exhaust gases pass through the cartridges 310 and the sorbent material may capture carbon dioxide from the exhaust gases.

In reference to FIG. 3, the cartridges 310 may also be connected in series via one or more coupler(s) 308. For example, the cartridge 311 may be connected in series with a cartridge 317 via a coupler 308. Likewise, the cartridge 313 may be connected in series with a cartridge 319 via a coupler 321. Similarly, the cartridge 313 may be connected in series with a cartridge 323 via a coupler 325. At the end of each series of cartridges, residual gases may pass through an entry port 312 of a last cartridge in the series and exit through an exit port 314 (described above). Connection in series allows for greater sorbent capacity such that when the sorbent material in one cartridge is saturated, the exhaust gases pass through to another cartridge where unsaturated sorbent material captures carbon dioxide from the remaining exhaust gases. The number of the cartridges in a series may be determined based on various factors such as (but not limited to) performance of the sorbent, operating conditions of a vehicle, etc., as would be known by a person of ordinary skill in the art.

As discussed previously, the carbon dioxide capture systems may be secured to a top, a bottom, a front, or a rear of a vehicle. Further, considering that the cartridges 310, the manifold 306, and the coupler(s) (e.g., couplers 308, 321, 325) will likely be in contact with exhaust gases that may be corrosive and/or of a high temperature, the cartridges 310, the manifold 306, and the coupler(s) 308, 321, 325 may be constructed using heat-resistant and corrosion-resistant material, including (but not limited to) metals, stainless steel, titanium, high-temperature plastics, and silicon carbide. Furthermore, the manifold 306, the coupler(s) 308, 321, 325, and the exhaust feeder 304 may also be constructed using a flexible material (e.g., PVC pipe), to adapt to the surfaces of different vehicles.

Although specific carbon dioxide capture systems utilizing cartridges in parallel and/or in series are discussed with respect to FIG. 3, any of a variety of carbon dioxide capture systems as appropriate to the requirements of a specific application can be utilized in accordance with embodiments of the invention. For example, in some embodiments, the carbon dioxide capture system may include only cartridges in parallel or only cartridges in series. Further, in some embodiments, the carbon dioxide capture system may include any number of cartridges in parallel or any number of cartridges in series. Cartridges in accordance with embodiments of the invention, are further described below.

### Cartridges

A cartridge may be used in a carbon dioxide capture system to capture the carbon dioxide from exhaust gases. As described above, a cartridge may be a container for sorbents or any other material that may be used to separate a target gas (e.g., CO₂) from the exhaust gases of a vehicle. In some embodiments, one or more cartridge(s) may be connected directly to an exhaust feeder via a manifold, as described above. In other embodiments, the cartridge may be placed inside a cargo box as a container for sorbent material, as described above.

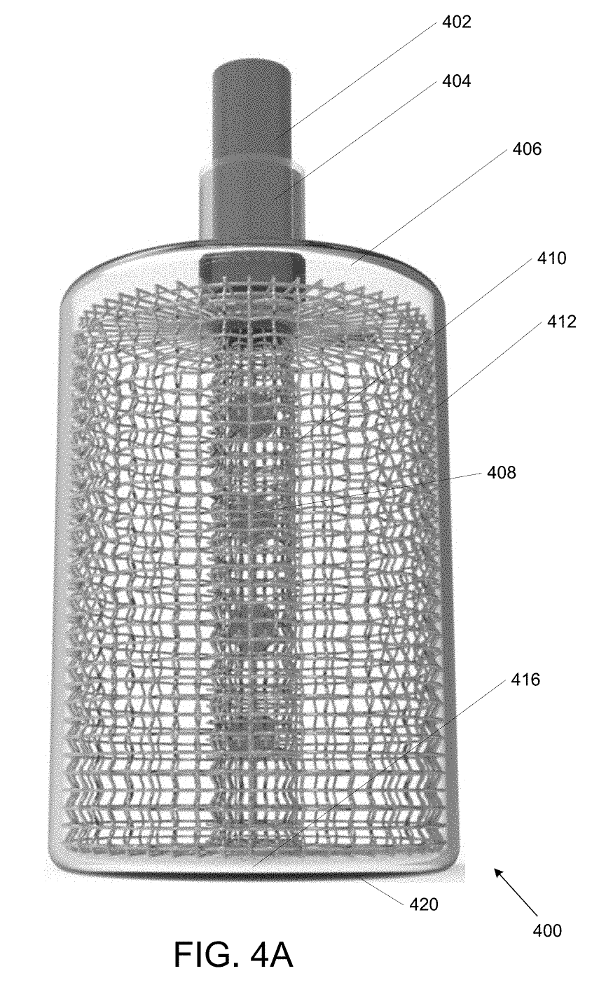

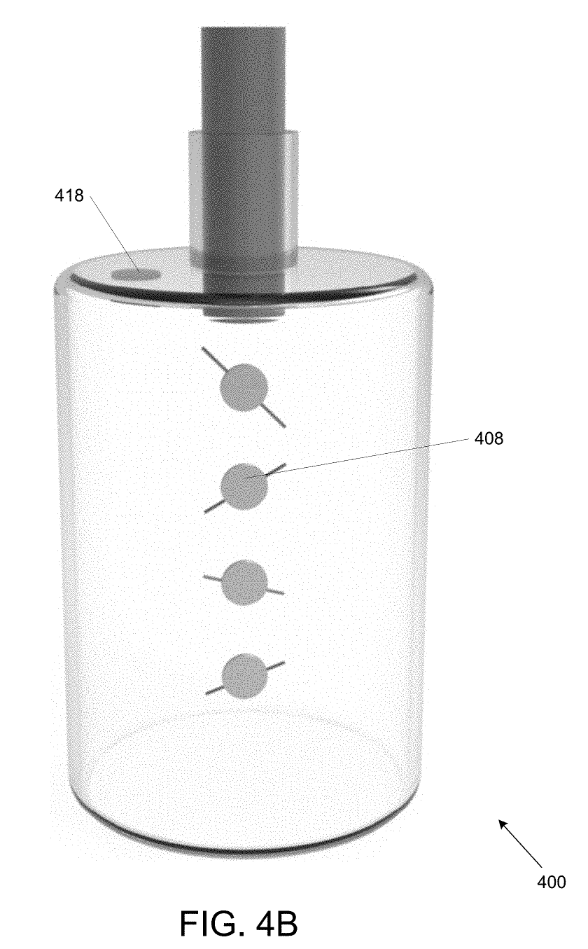

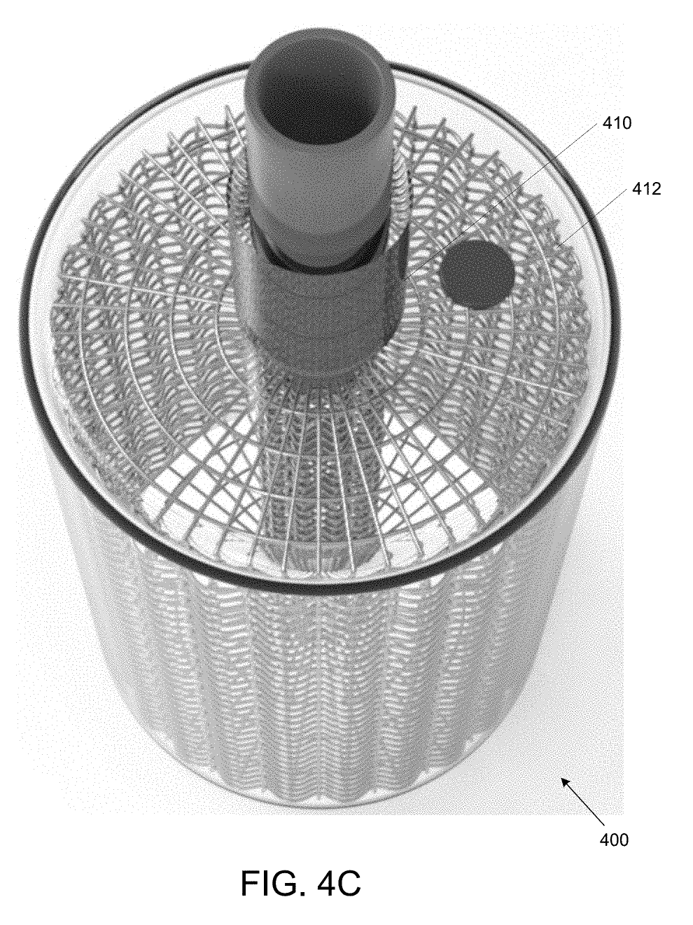

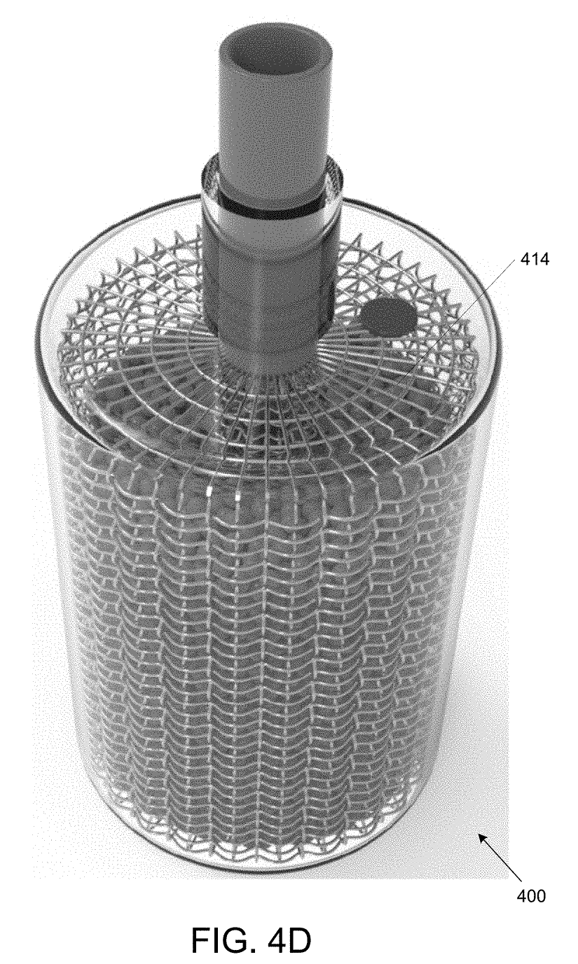

A diagram illustrating a cartridge comprising meshes in accordance with an embodiment of the invention is shown in FIG. 4A. In reference to FIG. 4A, a cartridge 400 may include a connection to a second end of the exhaust feeder 402, where the exhaust feeder receives exhaust gases from a tailpipe of a vehicle and allows for the exhaust gases to travel to into an interior 406 of the cartridge 400, as described above. A flange 404 may be used to secure the exhaust feeder 402 to the cartridge 406 using a number of locking, slotting, or other securing mechanisms known to one of ordinary skill in the art. Exhaust gases, once in the interior of the cartridge 406, may be redirected via gas deflector surfaces 408, which may be openings fixed to or movable relative to an internal mesh 410. The internal mesh 410 may be encased by an external mesh 412. Both the internal mesh 410 and the external mesh 412 may be of cylindrical shape and centered around the exhaust feeder 402, so that a predictable path for the exhaust gases through sorbent material (not shown in FIG. 4A) is generated. Together, the space between the internal mesh 410 and external mesh 412 may hold a sorbent material that captures carbon dioxide from redirected exhaust gases. In other embodiments, a plurality of mesh bags containing sorbent material that captures carbon dioxide from exhaust gases may be used instead of the internal mesh 410 and the external mesh 412. The mesh structure (e.g., in both the internal mesh 410 and the external mesh 412 and mesh bags) allows exhaust gases to pass through the sorbent material while still holding the sorbent material in place. Residual gases following carbon dioxide adsorption using sorbent material may exit via an exit port 416 located on the cartridge 400. When sorbent material is partial or fully saturated from carbon adsorption, a detachable lid 420 located on the cartridge 400 may be detached to allow for extraction and reattached for reinstallation of the sorbent material.

A diagram illustrating a cartridge without meshes in accordance with an embodiment of the invention is shown in FIG. 4B. The gas deflector surfaces 408, may be openings located inside the cartridge 400, as discussed above, and are shown in greater detail in FIG. 4B. The gas deflector surfaces 408 may be used to create various openings such that exhaust gases travel through a pre-defined path to the sorbent material. A locking mechanism 418, as would be known by a person of ordinary skill in the art, may be used to secure an exhaust feeder to the cartridge 400, as shown in FIG. 4B. Other mechanisms for securing an exhaust feeder to the cartridge 400 may include a slotting mechanism, as would be known by one of ordinary skill in the art.

A top-down perspective diagram illustrating a cartridge comprising meshes in accordance with an embodiment of the invention is shown in FIG. 4C. The internal mesh 410 and the external mesh 412 of the cartridge 400 are shown in greater detail. A top-down perspective diagram illustrating a cartridge comprising meshes and sorbent in accordance with an embodiment of the invention is shown in FIG. 4D. As discussed previously, a sorbent material 414 that captures carbon dioxide from exhaust gases may be placed in the cartridge 400, such as in between an internal mesh and an external mesh. In some embodiments, the sorbent material 414, as discussed above, may also be placed in mesh bags instead of an internal mesh and an external mesh.

In many embodiments, the cartridge 400 may be made of materials that are heat-resistant and corrosive-resistant, including metals, stainless steel, titanium, high-temperature plastics, and silicon carbide. The sorbent material 414 may comprise of any material that captures carbon dioxide (preferably at least 15 percent of mass in carbon dioxide captured), including metal-organic frameworks (MOFs), zeolites, silica, calcium oxides, organic-inorganic hybrids, alkylamines/amines, hydrotalcites, and activated carbons. A list of the commonly available sorbent material 414 is discussed in Table 1. Typically, different performance of the types of sorbent material 414 under various vehicle operating conditions should be expected. The sorbent material 414 may be of various size and shape, including pieces of sorbent in spherical shape formed together, such that it is held securely in between meshes or a mesh bag. In various embodiments, a mesh bag may be of any container with pore sizes that allow for inflow of gases while still holding the sorbent material 414. Various materials may be used in a mesh bag (e.g., nylon and cotton, among others). When the sorbent material 414 is partially or fully saturated with carbon dioxide, it may be removed, via the detachable lid 420 of the cartridge 400, and reinstalled following sorbent regeneration which allows for storage of carbon dioxide for biological carbon fixation. An example of biological carbon fixation is the use of carbon dioxide along with photosynthesis to achieve algae growth, with algae capable of being used in biofuels, biomass generation, wastewater treatment, and fertilizers.

Although specific cartridges are discussed with respect to FIGS. 4A-D, any of a variety of cartridges as appropriate to a specific application can be utilized in accordance with embodiments of the invention. Processes for reducing carbon dioxide emissions using carbon dioxide capture systems in accordance with embodiments of the invention are further discussed below.

### Reducing Carbon Dioxide Emissions Using Carbon Dioxide Capture Systems

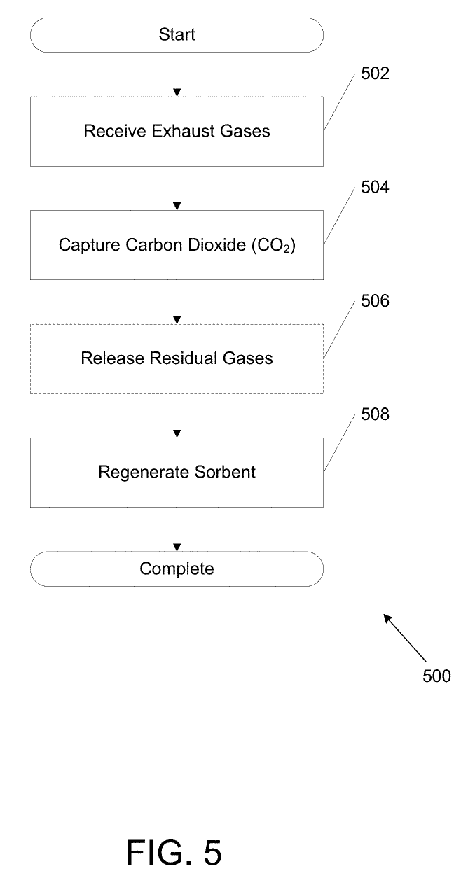

A carbon dioxide capture system may be utilized to reduce carbon dioxide emissions. A flowchart illustrating a process for reducing carbon dioxide emissions from a vehicle in accordance with an embodiment of the invention is shown in FIG. 5. The process 500 may include receiving (502) exhaust gases from a vehicle. For example, exhaust gases may be received (502) from a tailpipe of a vehicle via an exhaust feeder, as described above. In various embodiments, the exhaust gases may travel via the exhaust feeder into a cargo box via an input port, as described above. In such embodiments, the exhaust gases may pass through one or more cartridges located within the cargo box and carbon dioxide may be captured (504) using sorbent material. In some embodiments, the exhaust gases may travel via the exhaust feeder (and possibly a manifold and/or coupler) directly into the cartridges, and carbon dioxide may be captured (504) using sorbent material, as described above. In various embodiments, the process 500 may include the release (506) of residual gases via an exit port. In many embodiments, the process 500 may also include regenerating (508) the sorbent, as further described below.

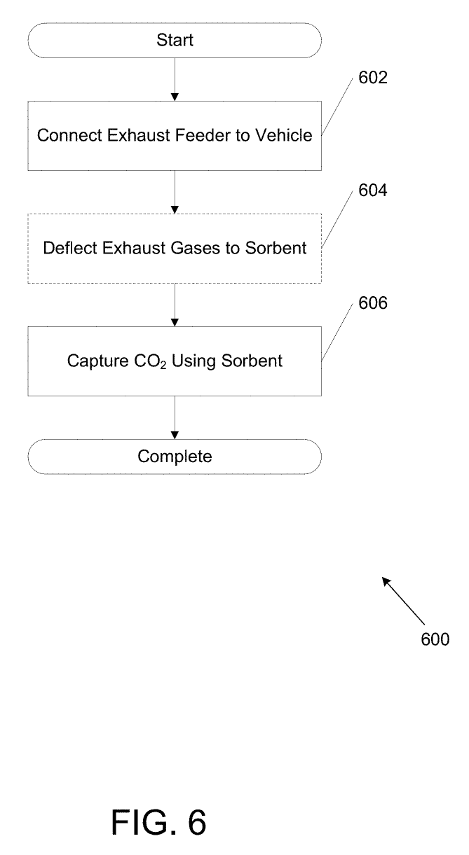

A flowchart illustrating a process for capturing CO₂ in accordance with an embodiment of the invention is shown in FIG. 6. The process 600 may include connecting (602) an exhaust feeder to a vehicle, as described above. For example, a first end of the exhaust feeder may be connected (602) to a tailpipe of the vehicle. As described above, the exhaust feeder, may include a first end connected to the tailpipe and a second end connected to the housing. In some embodiments, the process 600 may also include deflecting (604) exhaust gases to a sorbent material. For example, the carbon dioxide capture system may include one or more gas deflector surfaces (e.g., openings) for deflecting (604) the exhaust gases to the sorbent material. The process 600 may also include capturing (606) carbon dioxide using the sorbent, as described above.

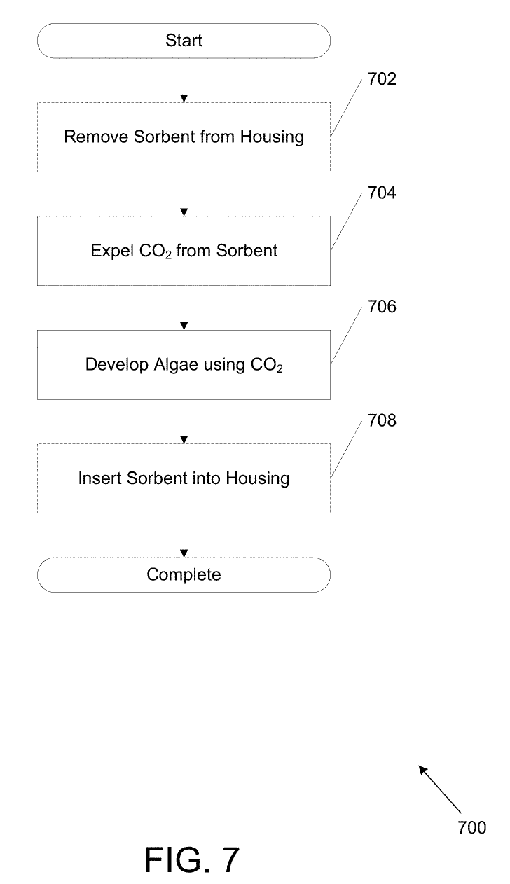

Regeneration of a sorbent allows for reuse of existing sorbent material and utilization of captured CO₂ for beneficial processes such as (but not limited to) algae development. In turn, algae development allows for generation of biofuels, biomass, utilities in wastewater treatment, and fertilizers. A flowchart illustrating a process for regenerating a sorbent in accordance with an embodiment of the invention is shown in FIG. 7. In some embodiments, the process 700 may include removing (702) a sorbent from the housing, as described above. For example, the sorbent may be contained in a cartridge that is held within the housing, as described above. In such embodiments, the sorbent may be removed (702) from the housing via a detachable section that releases the cartridge containing the sorbent. The process 700 may also include expelling (704) the carbon dioxide from the sorbent. For example, the carbon dioxide captured in the sorbent may be expelled (704) using elevated temperatures and/or creating a reduced pressured environment. For example, the sorbent may be microwaved at 100-130 degrees Celsius. Other methods, provided they elevate the temperature and/or reduce pressure (e.g., steaming and heating), may also be used to expel the carbon dioxide from the sorbent. The process 700 may also include developing (706) algae using the carbon dioxide. For example, the expelled carbon dioxide may be stored for developing algae (706) using a process known as biological carbon fixation (e.g., photosynthesis, among others). In many embodiments, algae growth may be used for biofuels, biomass generation, substances in wastewater treatment, and fertilizers, etc. In some embodiments, the process 700 may also include inserting (708) the sorbent back into the housing. For example, in some embodiments, the sorbent may be placed back to the cartridge. Further, in some embodiments, the cartridge may be inserted into the housing.

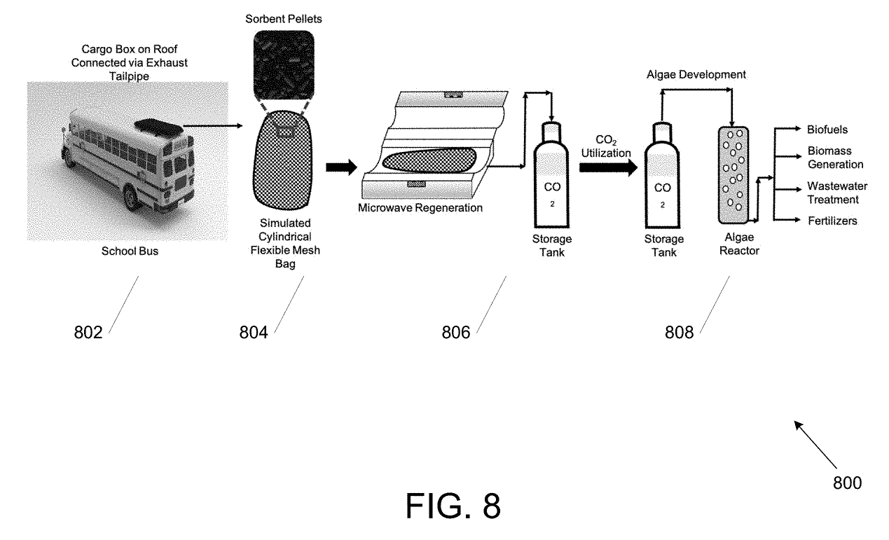

A flowchart illustrating a process for reducing CO₂ emissions from a vehicle in accordance with another embodiment of the invention is shown in FIG. 8. As described above, a carbon dioxide capture system may be utilized to capture carbon emissions from a vehicle. The process 800 may include receiving (802) exhaust gases, where a sorbent may capture carbon dioxide from the exhaust gases, as described above. In various embodiments, the process 800 may also include removing (804) the sorbent for servicing. For example, in some embodiments, the sorbent material may be removed from a housing of a cargo box via a detachable section of the housing after opening the cargo box. In many embodiments, the process 800 may also include regenerating (806) the sorbent, as described above. For example, the sorbent material may be heated (e.g., microwaving at 100-130 degrees Celsius) and/or applying reduced pressure to expel carbon dioxide captured in the sorbent material and thereby regenerating the sorbent. The process 800 may further include utilizing (808) the expelled carbon dioxide. For example, the expelled carbon dioxide may be stored in a storage tank, where the carbon dioxide may be used in algae development, as described above. For example, the algae development may utilize (808) an algae reactor for biological carbon fixation (e.g., photosynthesis, among others). Further, the algae may then be utilized for beneficial uses such as (but not limited to) generating biofuels, biomass, substances in wastewater treatment, fertilizers, etc.

Although specific process for reducing carbon emissions from a vehicle using carbon dioxide capture systems are discussed with respect to FIGS. 5-8, any of a variety of processes as appropriate to the requirements of a specific application can be utilized in accordance with embodiments of the invention.

While the above descriptions contain many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. An apparatus for capturing carbon emissions from a vehicle, comprising:
an exhaust feeder comprising a first end and a second end, wherein the first end of the exhaust feeder connects to the vehicle and receives exhaust gases emitted from the vehicle;
a housing connected to the second end of the exhaust feeder and secured to an exterior surface of the vehicle, wherein the housing receives the exhaust gases emitted from the vehicle via the exhaust feeder;
wherein the housing comprises:
at least one gas deflector surface that directs the exhaust gases to a sorbent material configured to capture carbon dioxide from the exhaust gases;
a detachable section that allows for removal and installation of the sorbent material; and
an exit port that allows for release of residual gases after the sorbent material captures carbon dioxide from the exhaust gases.

2. The apparatus of claim 1, wherein the first end of the exhaust feeder connects to the vehicle via a tailpipe of the vehicle.

3. The apparatus of claim 1, wherein the exhaust feeder is constructed using heat-resistant and corrosion-resistant materials.

4. The apparatus of claim 1, wherein the housing is constructed using heat-resistant and corrosion-resistant materials.

5. The apparatus of claim 1, wherein the housing is secured to a top, a bottom, or a rear of the vehicle.

6. The apparatus of claim 1, wherein the housing further comprises a plurality of mesh bags configured to hold the sorbent material while still allowing the exhaust gases directed from the gas deflector surfaces to pass through the sorbent material.

7. The apparatus of claim 1, wherein the apparatus further comprises a flange that secures the exhaust feeder to the housing using a locking mechanism.

8. The apparatus of claim 1, wherein the apparatus further comprises a flange that secures the exhaust feeder to the housing using a slotting mechanism.

9. The apparatus of claim 1, wherein the exhaust feeder is constructed using a flexible material.

10. The apparatus of claim 1, wherein the gas deflector surfaces are located in the housing and centered around the exhaust feeder.

11. A method for reducing carbon emissions from a vehicle, comprising:
receiving exhaust gases emitted from a vehicle at a first end of an exhaust feeder;
delivering exhaust gases from the first end of the exhaust feeder to a second end of the exhaust feeder;
transporting the exhaust gases from the second end of the exhaust feeder into a housing secured to an exterior surface of a vehicle;
directing the exhaust gases in the housing to a sorbent material using gas deflector surfaces;
adsorbing carbon dioxide from the exhaust gases using the sorbent material;
expelling residual gases using an exit port in the housing after the sorbent material captures carbon dioxide from the exhaust gases;
removing a section of the housing to allow for extraction of the sorbent material;
heating the sorbent material after extraction to regenerate the sorbent material;
storing carbon dioxide expelled from the sorbent material during heating of the sorbent material in a storage tank;
utilizing the carbon dioxide for algae development including usage in biofuels, biomass generation, wastewater treatment, and fertilizers; and
reinstalling the sorbent material in the housing after regeneration of the sorbent material from heating.

12. The method of claim 11, wherein the first end of the exhaust feeder connects to the vehicle via a tailpipe of the vehicle.

13. The method of claim 11, wherein the exhaust feeder is constructed using heat-resistant and corrosion-resistant materials.

14. The method of claim 11, wherein the housing is constructed using heat-resistant and corrosion-resistant materials.

15. The method of claim 11, wherein the housing is secured to a top, a bottom, or a rear of the vehicle.

16. The method of claim 11, wherein the housing further comprises a plurality of mesh bags configured to hold the sorbent material while still allowing the exhaust gases directed from the gas deflector surfaces to pass through the sorbent material.

17. The method of claim 11, wherein a flange secures the exhaust feeder to the housing using a locking mechanism.

18. The method of claim 11, wherein a flange secures the exhaust feeder to the housing using a slotting mechanism.

19. The method of claim 11, wherein the exhaust feeder is constructed using a flexible material.

20. The method of claim 11, wherein the gas deflector surfaces are located in the housing and centered around the exhaust feeder.
